(19) United States
(12) Patent Application Publication
(10) Pub. No.: US 2024/0335416 A1
(43) Pub. Date: Oct. 10, 2024

(54) GENOTYPE-SPECIFIC METHODS AND SYSTEMS FOR TREATMENT OF NEURODEGENERATIVE DISEASE

(71) Applicant: PhenoNet, Inc., Newton, MA (US)

(72) Inventor: David R. ELMALEH, Newton, MA (US)

(73) Assignee: PhenoNet, Inc., Newton, MA (US)

(21) Appl. No.: 18/626,002

(22) Filed: Apr. 3, 2024

Related U.S. Application Data
(60) Provisional application No. 63/494,064, filed on Apr. 4, 2023.

(51) Int. Cl. A61K 31/352; A61K 31/192

(52) U.S. Cl. CPC A61K 31/352; A61K 31/192

(57) ABSTRACT

The present disclosure provides methods of treatment of neurodegenerative disease in subjects in need thereof. A therapeutically effective dose of combination of a suitable mast cell stabilizer and NSAID may be administered to suitable subjects, wherein the suitability of a subject is determined by subject genotype. In certain embodiments, the combination therapy may comprise a combination of a cromolyn homolog salt and an NSAID, and suitable subjects are persons who are not carriers APOE ε4 variant of the APOE gene.

### CROSS-REFERENCE TO RELATED APPLICATIONS

This application claims priority to and the benefit of U.S. Provisional Application No. 63/494,064, filed on Apr. 4, 2023, which is hereby incorporated by reference in its entirety.

### FIELD OF INVENTION

The present disclosure relates to methods of treatment of neurodegenerative disease in suitable subjects in need thereof.

### BACKGROUND

Alzheimer's disease is a neurodegenerative disorder of the brain, characterized by deterioration of short-term memory formation and recall, deterioration of long-term memory recall, behavioral disturbances, disorientation, impairment of daily activities and life functions, loss of independence, and even death. As of the end of 2020, an estimated 55 million people worldwide live with some form of Alzheimer's disease. (See ALZHEIMER'S DISEASE INT'L, Numbers of people with dementia around the world, https://www.alzint.org/resource/numbers-of-people-with-dementia-worldwide/(30 Nov. 2020)). This number is predicted to approximately double every 20 years.

Alzheimer's disease is complicated. It is probably not “one” disease as such, but a spectrum or genus or diseases with similar causes and presentation. The causes are likely multifactorial. There is a consensus that Alzheimer's disease may be characterized by an accumulation of insoluble aggregates of amyloid-beta peptide (Aβ), such as Aβ oligomers. These aggregates or oligomers are associated with cell inflammatory response and are thought to bind to a surface receptor on neurons and change the structure of the neuronal synapse, thereby disrupting neuronal communication. Due to the tiny amounts of Aβ produced per day (estimated 22-27 ng/day throughout the brain) and accumulated for years (about 7-10 mg in brains of Alzheimer's disease subjects), this daily inflammatory response is virtually invisible and not associated with any major symptoms. In addition, tubulin-associated unit protein (“tau protein” or “t protein”) abnormalities are thought to play a role in the disease cascade leading to onset and progression of Alzheimer's disease. Hyperphosphorylated tau proteins are thought to pair with other threads of tau. Eventually, they form neurofibrillary tangles inside nerve cell bodies. When this occurs, the microtubules disintegrate, collapsing the neuron's transport system. This may result first in malfunctions in biochemical communication between neurons, and later in cell death.

In addition to a still evolving understanding of the causes and progression of Alzheimer's disease, including the importance of Aβ and tau proteins, some genetic predictors of Alzheimer's disease onset have been discovered. While Alzheimer's disease does not seem to be primarily hereditary in most cases, many people do carry a genetic predisposition for Alzheimer's disease. In some instances, a subject's genetic predisposition for Alzheimer's disease may be strong, and highly predictive that a person will eventually suffer from Alzheimer's disease.

Despite understanding certain features of Alzheimer's disease, as set forth above, effective treatments remain elusive. Promising drug candidates have failed, spurring greater urgency to investigate new targets. New therapies are greatly needed. Until now, investigators seeking drug candidates have not taken subject genotype into account, despite researchers knowing that genotype can strongly influence the expected courses of the disease. Because the causes of Alzheimer's disease are multifactorial and include at least some element of genetic predisposition, there is a great unmet need for more personalized methods of treatment tailored to a subject's genotype.

### SUMMARY OF THE INVENTION

In an aspect, the present disclosure relates to embodiments of methods of treatment of neurodegenerative disease in a subject in need thereof, the method comprising administering to the subject an effective dose of a combination of a mast cell stabilizer and a non-steroidal anti-inflammatory drug; wherein the subject is not a carrier for a neurodegenerative disease predisposing genotype.

In any embodiment of the method of treatment, the neurodegenerative disease may be Alzheimer's disease (“AD”). In any embodiment of the method of treatment, the method may treat ongoing neurodegenerative disease and/or prevent future onset of neurodegenerative disease. In any embodiment, the neurodegenerative disease may, at the time of treatment, be diagnosed or undiagnosed. The neurodegenerative disease may, at the time of treatment, may be early onset. In any embodiment, the mast cell stabilizer may be cromolyn, a cromolyn derivative, a cromolyn homolog, a salt of any of cromolyn, a cromolyn derivative, or a cromolyn homolog, or any combination thereof. In any embodiment, the salt may be a biocompatible salt. In any embodiment, the biocompatible cromolyn salt may be cromolyn sodium.

In any embodiment of the method of treatment, the subject may not be a carrier of any variant of Apolipoprotein E predisposing the subject to onset of neurodegenerative disease, including Alzheimer's disease. In any embodiment, the subject may not be a carrier of APOE ε4.

In any embodiment of the method of treatment, the cromolyn or biocompatible salt thereof may be formulated as a dry powder for inhalation. In any such embodiment, the cromolyn or biocompatible salt thereof may be formulated for inhalation as a dry powder of less than about 3 microns in particle size.

In any embodiment of the method of treatment, the cromolyn or biocompatible salt thereof may be administered at a dose of about 16 mg/day to about 20 mg/day.

### BRIEF DESCRIPTION OF THE DRAWINGS

FIG. 1 depicts an exemplary primary-structure diagram of a generic human ApoE protein, pointing out where certain differences exist in the common ApoE isoforms. While many described isoforms exist, by far the most prevalent are the ApoE2, ApoE3, and ApoE4 isoforms, which are encoded by the 82, 83 and 84 homologs of the APOE gene, respectively. The E2, E3, and E4 isoforms differ from one another at amino acid residues 112 and/or 158 (shown in FIG. 1 as gray circles). ApoE2 has cysteine residues at both 112 and 158; ApoE3 has a cysteine residue at 112 and an arginine residue at 158; and ApoE4 has arginine residues at both 112 and 158. ApoE protein has two structural domains: the N-terminal domain, which contains the receptor-binding region (at residues 136-150), and the C-terminal domain, which contains the lipid-binding region (at residues 244-272); the two domains are joined by a “hinge” region. Analyses have shown a significant positive correlation between carrying one or two alleles of ApoE4 and early onset of Alzheimer's disease.

FIG. 2 depicts a table of approximate frequency and average age of onset of Alzheimer's disease in persons not carrying any apoE ε4 alleles, persons heterozygous for apoE ε4, and persons homozygous for apoE ε4.

FIG. 3 depicts an exemplary diagram of amyloid-β protein clearance pathways. These include receptor-mediated uptake by neurons and glia, drainage into interstitial fluid or through the blood-brain barrier (BBB), and proteolytic degradation by insulin-degrading enzyme (IDE) and neprilysin (NEP). Impaired clearance of Aβ can cause Aβ accumulation in brain parenchyma, leading to formation of neurotoxic Aβ oligomers and amyloid plaques. Aβ accumulation in the perivascular region leads to cerebral amyloid angiopathy (CAA), which disrupts blood vessel function. ApoE is primarily synthesized by astrocytes and microglia, and is lipidated by the ATP-binding cassette A1 (ABCA1) transporter to form lipoprotein particles. Lipidated ApoE binds to soluble Aβ and facilitates Aβ uptake through cell surface receptors, including low-density lipoprotein receptor-related protein 1 (LRP1), low-density lipoprotein receptor (LDLR), heparan sulphate proteoglycan (HSPG) 175, and HSPG177 in a manner that may depend on the ApoE isoform and its level of lipidation. ApoE facilitates binding and internalization of soluble Aβ by glial cells, disrupts Aβ clearance at the BBB in an isoform-dependent manner (ApoE4>ApoE3>ApoE2) and influences CAA pathogenesis. As used in FIG. 3, LXR refers to liver X receptor.

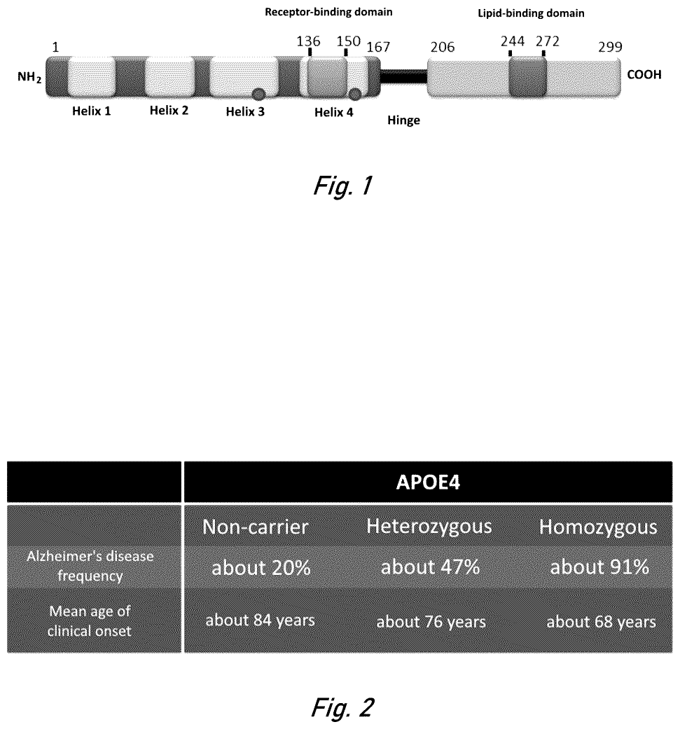

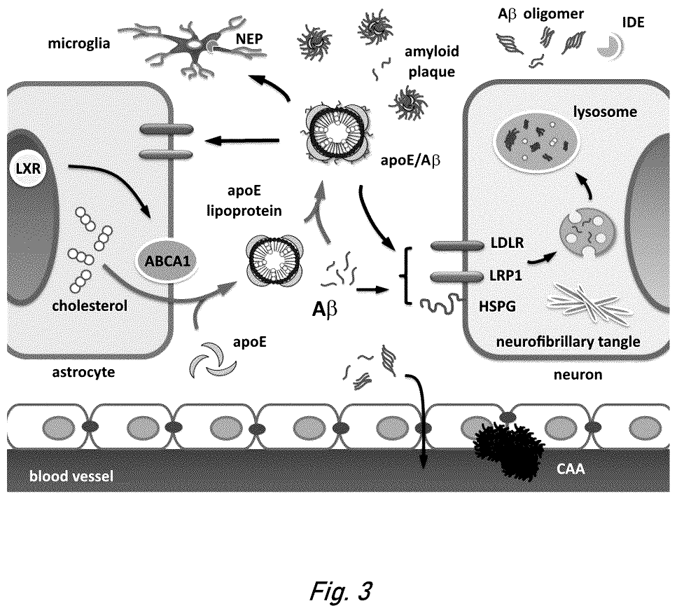

### DETAILED DESCRIPTION

The present disclosure provides methods of treatment of neurodegenerative disease in subjects in need thereof.

### Failures to Develop Safe and Effective Neurodegenerative Disease Treatments.

Neurodegenerative diseases such as Alzheimer's disease (AD) have had the attention of the medical and pharmaceutical industries since at least the 19th century. Basic science and treatment avenues have come a long way in the 21st century, but safe and effective treatment options remain nascent at best. There are almost no treatments for neurodegenerative disease despite an estimated 55 million afflicted persons worldwide. Beside the controversial 2021 U.S. Food and Drug Administration approval of aducanumab, the most recent medication to earn FDA approval for treatment of Alzheimer's disease was memantine in 2003, and while memantine manages some symptoms, current therapies offer no hope of reversal of disease progression.

Why do cures for neurodegenerative disease not exist yet? One problem is that it is challenging to design and implement clinical studies that have an appropriate clinical benefit endpoint. Researchers lack the necessary diagnosis and quantitative measurement tools to describe and evaluate disease progression. Further, the causes of neurodegenerative disease are multifactorial, and a measure of one factor may overlook others. Thus, a clinical trial may appear, according to a selected readout, appear to be unable to refute the null hypothesis. But if designed and interpreted from another perspective, the same study may show efficacy, e.g., for some subpopulations of clinical subjects.

Discovering safe and effective therapies for neurodegenerative disease is challenging because, among other reasons, it is a complicated health issue with over 50 underlying causal factors, rather than a single diagnosable illness. Alzheimer's disease is the most prevalent form of neurodegenerative disease, and is better understood as a generic term for progressive neurodegenerative disorders presenting deterioration of short-term memory formation and recall, progressive decline of long-term memory recall, behavioral disturbances, disorientation, impairment of daily activities and life functions, lost of independence, and even death. Alzheimer's disease accounts for 60 to 70 percent of all dementia cases, and has been the focus of most pharmacological therapies research. The science behind Alzheimer's disease drug development is vast, conceptually based on a solid foundation of basic science, and substantially validated in vitro and in vivo in animal models. Yet, in practice, the results of clinical trials since the 1990s have consistently failed to comport with the underlying hypothesis and rationale, and fail to reach the predetermined clinical benefit endpoint.

There are many obstacles to developing promising neurodegenerative disease therapies. Among these include, principally, (1) inadequate basic-science understanding of the complex physiology of neurodegenerative diseases; (2) lack of pragmatic primary efficacy measures; and (3) overreliance on biomarkers as a surrogate for clinical efficacy.

As to the first obstacle, the inadequate basic-science understanding of the complex physiology of neurodegenerative diseases, while the research community has come a long way in understanding of the biology behind the illnesses and the multifactorial causes of the diseases, still a lot is yet to be elucidated. For instance, scientists do not yet fully understand what controls the toxic build-up of amyloid plaques and tau-protein tangles in Alzheimer's patients' brains, the reasons and effects underlying genetic predispositions, environmental impact, or why the illness advances at varying rates in various individuals. However, researchers developed solid scientific approaches for their drug development and validated them in vitro and animal models; therefore, it is unlikely that the failure is due only to the science behind it.

As to the lack of pragmatic primary efficacy measures, the study designs and the tools required to accurately diagnose and evaluate disease progression remain the most significant challenge. Most, if not all, clinical trial designs and the available measuring tools (i.e., CDR-SB and all ADS-cogs) are disposed to a higher degree of failure instead of the readout of substantial clinical benefit. The reasons are associated with many imperfect measuring tools and the multifactorial causes of disease progression. For example, the Alzheimer's Clinical Dementia Rating (CDR) readout is vulnerable to many variables, such as mood or caretaker assessment. These tools do not represent an absolute measurable change as in the case of, e.g., cancer, where a difference in tumor size or its spread is measurable. The available Alzheimer's disease CDR measurement is semi-quantitative at best.

Further, the likelihood that any new drug would significantly reverse the chronic cognition and functional decline of a compromised brain of an Alzheimer's disease patient is far smaller than stabilizing or slowing down its progression; therefore, cognitive scales like CDR-SB, for example, which are more likely to measure significant declines versus stable, slow down, or slight improvement in Alzheimer's disease may not be optimum endpoints for many novel therapies. In other words, for a disease that takes many years to present clinically, it may not be realistic to expect therapeutic intervention to achieve measurable reversal. Assessing the effects of a treatment in a pre-symptomatic population is even more complicated. Many cognitive scales which are useful outcome measures in established neurodegenerative disease, may not be sensitive enough in the pre-clinical stages of the disease. (See Vellas B et al., Endpoints for Pre-Dementia Alzheimer's Disease Trials: A Report From the EU US CTAD Task Force, J PREV ALZHEIMERS DIS 2015 June; 2 (2): 128-35.) Other factors affecting trial results are the considerable variation in subjects and within subjects due to disease heterogeneity and covariates such as age, rate of progression diagnosis, and other entry criteria.

Finally, as to the overreliance on biomarkers as a surrogate for clinical efficacy, biomarkers cannot replace clinical symptoms benefits as an efficacy readout. Biomarkers may help predict the direction of drug development to achieve efficacy, but protein measurements simply cannot tell you what a patient is experiencing cognitively. Some clinical studies, nevertheless, has relied on an accepted changes in biomarker levels (e.g., amyloid-β and/or tau) as surrogate clinical endpoints, without sufficient evidence of confirmatory changes in cognitive/functional/global neurological performance when deciding to advance to the pivotal trial. This is despite the fact that these performance tests are the primary clinical measure in phase III clinical trials for dementia-type neurodegenerative diseases. Ultimately, these trials failed as clinical efficacy endpoints were not met. (See Kim C K et al., Alzheimer's Disease: Key Insights from Two Decades of Clinical Trial Failures, J ALZHEIMERS DIS 2022 May 87 (1) 83-100.)

The road to a cure for neurodegenerative disease- or, more likely, a safe and effective therapy capable of slowing disease progression and positively affecting the quality-of-life of patients and their families—is complex. However, it is achievable.

Neurodegenerative diseases such as Alzheimer's disease often are chronic illnesses with an insidious, protracted onset. Alzheimer's typically presents with a long preclinical and prodromal phases of about 20 years before the onset of the noticeable symptoms. The appearance of symptoms reflects a cerebral network that is already compromised and associated with massive neuronal loss and a neuroinflammatory response that is fast spreading. Unfortunately, with the present state of the science, we cannot diagnose Alzheimer's 20 years before the first symptoms appear.

Currently, accessible drugs, like those that block the actions of an enzyme that decreases an active chemical messenger for memory in the brain (acetylcholinesterase inhibitors) or prevent the toxic effects of another messenger, glutamate (memantine), provide only temporary relief from symptoms. Recently approved biologic aducanumab is monoclonal Ig1 antibody that binds to the amyloid-β proteins and is expected to lead to dose-dependent removal of β-amyloid pathology, though the evidence of clinical efficacy is arguably insufficient. (See Tampi R R et al., Aducanumab: Evidence from Clinical Trial Data and Controversies, DRUGS CONTEXT 2021 Oct. 4; 10:2021-7-3.) However, emerging therapies will address the underlying biology to halt or slightly reverse the disease process.

Additionally, because of the chronic nature of Alzheimer's disease and its multifactorial causality, the newly developed therapies are addressing the treatment of more than one pathway, for example, combining treatment that inhibits plaque and tangles or their removal and at the same time halt or treats the neuroinflammatory spread, or enhancing microglia and macrophage clearing damaged neurons and toxic debris. Unfortunately, the lengthy and expensive studies that last years before a proven therapy can be reached hamper investment excitement. In contrast, the development of drugs wherein researchers can quickly determine whether the medicine is effective, for example antibiotics, becomes more attractive. The tech giant Pfizer decided to stop its research for the cure for Alzheimer's disease and Parkinson's disease after many costly unsuccessful attempts and develop therapies for easier drug development and approval. With all that said, the potential unmet need for neurodegeneration would continue to attract innovative approaches for treatment by the government and industry.

Responding to the long-felt challenges and industry failures described supra, the present disclosure provides methods of treating neurodegenerative disease that may be tailored according to certain multivariate factors. In particular, clinical study and treatment designs for the treatment of neurodegenerative disease may be tailored according to patient genotype, as elaborated infra.

### Therapeutic Agents.

Featured herein are methods of treating or preventing a neurodegenerative disease in a subject in need thereof comprising administering to the subject a therapeutically effective amount of at least a mast cell stabilizer and a non-steroidal anti-inflammatory drug (NSAID) in a subject who is not a carrier for a predisposing genotype.

The mast cell stabilizer may be any biocompatible salt of cromolyn, a cromolyn derivative, or a cromolyn homolog. Cromolyn homologs may be generally defined according to formula I, below:

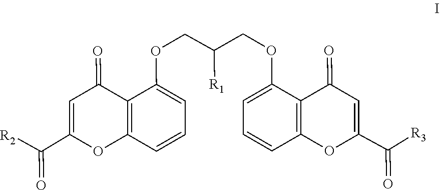

wherein R₁, R₂, and R₃ may be the same or different from each other, and wherein each may be any of —H, —OH, —F, —Cl, —Br, —I, —NH₂, —NO₂, —SH, —C(O)NH₂, —CN, an alkyl, a haloalkyl, an aryl, a haloaryl, an ether, an ester, or an aldehyde.

The NSAID is an anti-inflammatory compound not in the steroid class of compounds, which tends to reduce pain, decrease inflammation, decrease fever, and prevent blood clotting. NSAIDs generally inhibit cyclooxygenase enzymes COX-1 and/or COX-2. Exemplary NSAIDS include, but are not limited to, acetylsalicylic acid, celecoxib, dexibuprofen, dexketoprofen, diclofenac, diflunisal, droxicam, etodolac, fenoprofen, figwort, flufenamic acid, flurbiprofen, hyperforin, ibuprofen, indomethacin, isoxicam, ketoprofen, ketorolac, licofelone, lornoxicam, loxoprofen, meclofenamic acid, mefenamic acid, meloxicam, nabumetone, naproxen, oxaprozin, piroxicam, salsalate, sulindac, tenoxicam, tolmetin, and tolfenamic acid.

### Genetic Factors.

As early as the early 1990s, researchers noticed a correlation with certain genotypes to onset of Alzheimer's disease. (See, e.g., Pericak-Vance M. A., et al. (1991) Linkage studies in familial Alzheimer's disease: evidence for chromosome 19 linkage. AM. J. HUM. GENET. 48, 1034-50.) Further research discovered that the main predictor of Alzheimer's disease was genotype for the cholesterol transport protein Apolipoprotein E (apoE). (See Strittmatter W. J., et al., Apolipoprotein E: high-avidity binding to β-amyloid and increased frequency of type 4 allele in late-onset familial Alzheimer's disease. PROC. NATL. ACAD. SCI. USA 1993; 90, 1977-81.)

ApoE is one of several classes of apolipoproteins (including apoA, apoB, apoD, and apoJ) responsible for transporting lipids within and between cells. ApoE is an important cholesterol transport protein, mainly synthesized in the liver parenchymal cells, but also in adrenal gland, brain, fat, kidney, lung, and spleen. ApoE is polymorphic, existing in three major isoforms, with the major protein isoforms denoted as “apoE2”, “apoE3”, and “apoE4”. The corresponding gene variants are denoted as APOE ε2, APOE ε3, and APOE ε4, respectively. The APOE gene is located on the long arm of chromosome 19, contains four exons and three introns, and is about 3,639 bp long. Since a person carries two copies of genes on somatic chromosomes, a person's APOE genotype may be denoted as ε2/ε2; ε2/ε3; ε2/ε4; ε3/ε3; ε3/ε4; or ε4/ε4. Other apoE variants exist, but are considered very rare. Of the three major isoforms, the distribution of alleles in the general population is about 8% ε2, 78% ε3, and 14% ε4. Around 86% of the general population are not carriers of APOE ε4.

The APOE ε2 is underrepresented in cases of late-onset Alzheimer's disease. The APOE ε4 variant is significantly overrepresented in cases of late-onset Alzheimer's disease. This result is highly reliable, and has been substantiated across ethnic and racial groups, and for both early-onset and late-onset Alzheimer's disease. While about 14% of the general population carries APOE ε4, this number increases to around 40% of people diagnosed with Alzheimer's disease.

### Suitable Treatment Subjects.

For the present method, it was surprisingly and unexpectedly discovered in human clinical trials that certain combinations of therapeutic agents had a significantly greater effect on non-carriers of APOE ε4.

It was hypothesized that for subjects who are carriers of APOE ε4, the mechanism of action for certain therapies may be overwhelmed by the effect of the apoE4 protein variant on Alzheimer's disease onset and progression. Accordingly, the combination therapy of the present disclosure was discovered to be most effective in non-carriers of APOE ε4.

### Definitions

As used herein, the following terms and phrases should have the meanings provided below.

The indefinite articles “a” and “an,” as used herein in the specification and in the claims, unless clearly indicated to the contrary, should be understood to mean “at least one.”

For purposes of the present disclosure, an “amyloidosis-associated condition” is a disease that is associated with amyloid deposition and can include but not be limited to Alzheimer's disease, idiopathic myeloma, amyloid polyneuropathy, amyloid cardiomyopathy, systemic senile amyloidosis, amyloid polyneuropathy, hereditary cerebral hemorrhage with amyloidosis, Down's syndrome, Scrapie, medullary carcinoma of the thyroid, isolated atrial amyloid, β2-microglobulin amyloid in dialysis patients, inclusion body myositis, β2-amyloid deposits in muscle wasting disease, and Islets of Langerhans diabetes Type II insulinoma. Type 2 diabetes mellitus, hereditary cerebral hemorrhage amyloidosis (Dutch), amyloid A (reactive), secondary amyloidosis, familial Mediterranean fever, familial amyloid nephropathy with urticaria and deafness (Muckle-wells Syndrome), amyloid lambda L-chain or amyloid kappa L-chain (idiopathic, myeloma or macroglobulinemia-associated) A beta 2M (chronic hemodialysis), ATTR (familial amyloid polyneuropathy (Portuguese, Japanese, Swedish)), familial amyloid cardiomyopathy (Danish), isolated cardiac amyloid, systemic senile amyloidoses, AIAPP or amylin insulinoma, atrial naturetic factor (isolated atrial amyloid), procalcitonin (medullary carcinoma of the thyroid), gelsolin (familial amyloidosis (Finnish)), cystatin C (hereditary cerebral hemorrhage with amyloidosis (Icelandic)), AApo-A-1 (familial amyloidotic polyneuropathy-Iowa), AApo-A-II (accelerated senescence in mice), head injuries (traumatic brain injury), dementia, fibrinogen-associated amyloid; and Asor or Pr P-27 (scrapie, Creutzfeld Jacob disease, Gertsmann-Straussler-Scheinker syndrome, bovine spongiform encephalitis) or in cases of persons who are homozygous for the apolipoprotein E4 allele, and the condition associated with homozygosity for the apolipoprotein E4 allele or Huntington's disease.

“Amyloidosis” is a condition characterized by the accumulation of various insoluble, fibrillar proteins in the tissues of a patient. An amyloid deposit is formed by the aggregation of amyloid proteins, followed by the further combination of aggregates and/or amyloid proteins.

Many forms of amyloidosis exist, and the disease can be classified into four groups: primary amyloidosis, secondary amyloidosis, hereditary amyloidosis, and amyloidosis associated with normal aging. Primary amyloidosis (light chain amyloidosis) occurs with abnormalities of plasma cells, and some people with primary amyloidosis also have multiple myeloma (cancer of the plasma cells). Typical sites of amyloid buildup in primary amyloidosis are the heart, lungs, skin, tongue, thyroid gland, intestines, liver, kidneys, and blood vessels. Secondary amyloidosis may develop in response to various diseases that cause persistent infection or inflammation, such as tuberculosis, rheumatoid arthritis, and familial Mediterranean fever. Typical sites of amyloid buildup in secondary amyloidosis are the spleen, liver, kidneys, adrenal glands, and lymph nodes. Hereditary amyloidosis has been noted in some families, particularly those from Portugal, Sweden, and Japan. The amyloid-producing defect occurs because of mutations in specific proteins in the blood. Typical sites for amyloid buildup in hereditary amyloidosis are the nerves, heart, blood vessels, and kidneys.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

The terms “augmentation” or “augment” refer to combinations where one of the compounds increases or enhances therapeutic effects of another compound or compounds administered to a patient. In some instances, augmentation can result in improving the efficacy, tolerability, or safety, or any combination thereof, of a particular therapy.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures (M.P.E.P.), Section 2111.03.

The definition of each expression, e.g., alkyl, m, n, and the like, when it occurs more than once in any structure, is intended to be independent of its definition elsewhere in the same structure.

A comprehensive list of the abbreviations utilized by organic chemists of ordinary skill in the art appears in the first issue of each volume of the Journal of Organic Chemistry; this list is typically presented in a table entitled Standard List of Abbreviations.

The terms “hydroxy” and “hydroxyl” refer to the group —OH.

The term “oxo” refers to the group ═O.

The term “carboxylate” or “carboxyl” refers to the group —COO— or —COOH.

The term “cyano” refers to the group —CN.

The term “nitro” refers to the group —NO₂.

The term “amino” refers to the group —NH₂.

The term “acyl” or “aldehyde” refers to the group —C(═O)H.

The term “amido” or “amide” refers to the group —C(O)NH₂.

The term “aminoacyl” or “acylamino” refers to the group —NHC(O)H.

The term “thiol” refers to the group —SH.

The term “thioxo” refers to the group ═S.

The term “sulfinyl” refers to the group —S(—O) H.

The term “sulfonyl” refers to the group —SO₂H.

The term “sulfonylamido” or “sulfonamide” refers to the group —SO₂NH₂.

The term “sulfonate” refers to the group SO₃H and includes groups having the hydrogen replaced with, for example a C₁-C₆ alkyl group (“alkylsulfonate”), an aryl (“arylsulfonate”), an aralkyl (“aralkylsulfonate”) and so on. C₁-C₃ sulfonates are preferred, such as for example, SO₃Me, SO₃Et and SO₃Pr.

The term “isomers”, as used herein, refer to stereoisomers, diastereomers, enantiomers and tautomers. “Tautomers” may be isomers that are readily interconvertable by rapid equilibrium. For example, carbonyl compounds that have a hydrogen on their alpha-carbon are rapidly interconverted with their corresponding enols.

As used herein, the terms “alkyl”, “alkenyl”, and the prefix “alk-” are inclusive of straight chain groups and branched chain groups and cyclic groups, e.g., cycloalkyl and cycloalkenyl. Unless otherwise specified, these groups contain from 1 to 20 carbon atoms, with alkenyl groups containing from 2 to 20 carbon atoms. In some embodiments, these groups have a total of at most 10 carbon atoms, at most 8 carbon atoms, at most 6 carbon atoms, or at most 4 carbon atoms. Cyclic groups can be monocyclic or polycyclic and preferably have from 3 to 10 ring carbon atoms. Exemplary cyclic groups include cyclopropyl, cyclopropylmethyl, cyclopentyl, cyclohexyl, adamantyl, and substituted and unsubstituted bornyl, norbornyl, and norbornenyl.

The term “heterocyclic” includes cycloalkyl or cycloalkenyl non-aromatic rings or ring systems that contain at least one ring heteroatom (e.g., O, S, N).

Unless otherwise specified, “alkylene” and “alkenylene” are the divalent forms of the “alkyl” and “alkenyl” groups defined above. The terms, “alkylenyl” and “alkenylenyl” are used when “alkylene” and “alkenylene”, respectively, are substituted. For example, an arylalkylenyl group comprises an alkylene moiety to which an aryl group is attached.

The term “haloalkyl” is inclusive of groups that are substituted by one or more halogen atoms, including perfluorinated groups. This is also true of other groups that include the prefix “halo-”. Examples of suitable haloalkyl groups are difluoromethyl, trifluoromethyl, and the like. “Halogens” are elements including chlorine, bromine, fluorine, and iodine.

The term “aryl” as used herein includes monocyclic or polycyclic aromatic hydrocarbons or ring systems. Examples of aryl groups include phenyl, naphthyl, biphenyl, fluorenyl and indenyl. Aryl groups may be substituted or unsubstituted. Aryl groups include aromatic annulenes, fused aryl groups, and heteroaryl groups. Aryl groups are also referred to herein as aryl rings.

Unless otherwise indicated, the term “heteroatom” refers to the atoms O, S, or N.

The term “heteroaryl” includes aromatic rings or ring systems that contain at least one ring heteroatom (e.g., O, S, N). In some embodiments, the term “heteroaryl” includes a ring or ring system that contains 2 to 12 carbon atoms, 1 to 3 rings, 1 to 4 heteroatoms, and O, S, and/or N as the heteroatoms. Suitable heteroaryl groups include furyl, thienyl, pyridyl, quinolinyl, isoquinolinyl, indolyl, isoindolyl, triazolyl, pyrrolyl, tetrazolyl, imidazolyl, pyrazolyl, oxazolyl, thiazolyl, benzofuranyl, benzothiophenyl, carbazolyl, benzoxazolyl, pyrimidinyl, benzimidazolyl, quinoxalinyl, benzothiazolyl, naphthyridinyl, isoxazolyl, isothiazolyl, purinyl, quinazolinyl, pyrazinyl, 1-oxidopyridyl, pyridazinyl, triazinyl, tetrazinyl, oxadiazolyl, thiadiazolyl, and so on.

The terms “arylene” and “heteroarylene” are the divalent forms of the “aryl” and “heteroaryl” groups defined above. The terms “arylenyl” and “heteroarylenyl” are used when “arylene” and “heteroarylene”, respectively, are substituted. For example, an alkylarylenyl group comprises an arylene moiety to which an alkyl group is attached.

The term “fused aryl ring” includes fused carbocyclic aromatic rings or ring systems. Examples of fused aryl rings include benzo, naphtho, fluoreno, and indeno.

The term “annulene” refers to aryl groups that are completely conjugated monocyclic hydrocarbons. Examples of annulenes include cyclobutadiene, benzene, and cyclooctatetraene. Annulenes present in an aryl group will typically have one or more hydrogen atoms substituted with other atoms such as carbon.

When a group is present more than once in any formula or scheme described herein, each group (or substituent) is independently selected, whether explicitly stated or not. For example, for the formula —C(O)NR₂ each of the two R groups is independently selected.

As a means of simplifying the discussion and the recitation of certain terminology used throughout this application, the terms “group” and “moiety” are used to differentiate between chemical species that allow for substitution or that may be substituted and those that, in the particular embodiment of the invention, do not so allow for substitution or may not be so substituted. Thus, when the term “group” is used to describe a chemical substituent, the described chemical material includes the unsubstituted group and that group with nonperoxidic O, N, S, Si, or F atoms, for example, in the chain as well as carbonyl groups or other conventional substituents. Where the term “moiety” is used to describe a chemical compound or substituent, only an unsubstituted chemical material is intended to be included. For example, the phrase “alkyl group” is intended to include not only pure open chain saturated hydrocarbon alkyl substituents, such as methyl, ethyl, propyl, tert-butyl, and the like, but also alkyl substituents bearing further substituents known in the art, such as hydroxy, alkoxy, alkylsulfonyl, halogen atoms, cyano, nitro, amino, carboxyl, etc. Thus, “alkyl group” includes ether groups, haloalkyls, nitroalkyls, carboxyalkyls, hydroxyalkyls, sulfoalkyls, etc. On the other hand, the phrase “alkyl moiety” is limited to the inclusion of only pure open chain saturated hydrocarbon alkyl substituents, such as methyl, ethyl, propyl, tert-butyl, and the like.

The invention is inclusive of the compounds described herein (including intermediates) in any of their pharmaceutically acceptable forms, including isomers (e.g., diastereomers and enantiomers), tautomers, salts, solvates, polymorphs, prodrugs, and the like. In particular, if a compound is optically active, the invention specifically includes each of the compound's enantiomers as well as racemic mixtures of the enantiomers. It should be understood that the term “compound” includes any or all of such forms, whether explicitly stated or not (although at times, “salts” are explicitly stated).

“Pharmaceutically acceptable” as used herein means that the compound or composition or carrier is suitable for administration to a subject to achieve the treatments described herein, without unduly deleterious side effects in light of the necessity of the treatment.

The term “therapeutically effective amount” or “pharmaceutically appropriate dosage”, as used herein, refers to the amount of the compounds or dosages that will elicit the biological or medical response of a subject, tissue or cell that is being sought by the researcher, veterinarian, medical doctor or other clinician.

As used herein, “pharmaceutically-acceptable carrier” includes any and all dry powder, solvents, dispersion media, coatings, antibacterial and antifungal agents, isotonic agents, absorption delaying agents, and the like. Pharmaceutically-acceptable carriers are materials, useful for the purpose of administering the compounds in the method of the present invention, which are preferably non-toxic, and may be solid, liquid, or gaseous materials, which are otherwise inert and pharmaceutically acceptable, and are compatible with the compounds of the present invention. Examples of such carriers include oils such as com oil, buffers such as PBS, saline, polyethylene glycol, glycerin, polypropylene glycol, dimethylsulfoxide, an amide such as dimethylacetamide, a protein such as albumin, and a detergent such as Tween 80, mono- and oligopolysaccharides such as glucose, lactose, cyclodextrins and starch.

The term “administering” or “administration”, as used herein, refers to providing the compound or pharmaceutical composition of the invention to a subject suffering from or at risk of the diseases or conditions to be treated or prevented.

The term “systemic delivery”, as used herein, refers to any suitable administration methods which may delivery the compounds in the present invention systemically. In one embodiment, systemic delivery may be selected from the group consisting of oral, parenteral, intranasal, inhaler, sublingual, rectal, and transdermal administrations.

A route of administration in pharmacology and toxicology is the path by which a drug, fluid, poison, or other substance is taken into the body. Routes of administration may be generally classified by the location at which the substance is applied. Common examples may include oral and intravenous administration. Routes can also be classified based on where the target of action is. Action may be topical (local), enteral (system-wide effect, but delivered through the gastrointestinal tract), or parenteral (systemic action, but delivered by routes other than the GI tract).

A topical administration emphasizes local effect, and substance is applied directly where its action is desired. Sometimes, however, the term topical may be defined as applied to a localized area of the body or to the surface of a body part, without necessarily involving target effect of the substance, making the classification rather a variant of the classification based on application location. In an enteral administration, the desired effect is systemic (non-local), substance is given via the digestive tract. In a parenteral administration, the desired effect is systemic, and substance is given by routes other than the digestive tract.

The examples for topical administrations may include epicutaneous (application onto the skin), e.g., allergy testing or typical local anesthesia, inhalational, e.g. asthma medications, enema, e.g., contrast media for imaging of the bowel, eye drops (onto the conjunctiva), e.g., antibiotics for conjunctivitis, ear drops, such as antibiotics and corticosteroids for otitis externa, and those through mucous membranes in the body.

Enteral administration may be administration that involves any part of the gastrointestinal tract and has systemic effects. The examples may include those by mouth (orally), many drugs as tablets, capsules, or drops, those by gastric feeding tube, duodenal feeding tube, or gastrostomy, many drugs and enteral nutrition, and those rectally, various drugs in suppository.

The examples for parenteral administrations may include intravenous (into a vein), e.g. many drugs, total parenteral nutrition intra-arterial (into an artery), e.g., vasodilator drugs in the treatment of vasospasm and thrombolytic drugs for treatment of embolism, intraosseous infusion (into the bone marrow), intra-muscular, intracerebral (into the brain parenchyma), intracerebroventricular (into cerebral ventricular system), intrathecal (an injection into the spinal canal), and subcutaneous (under the skin). Among them, intraosseous infusion is, in effect, an indirect intravenous access because the bone marrow drains directly into the venous system. Intraosseous infusion may be occasionally used for drugs and fluids in emergency medicine and pediatrics when intravenous access is difficult.

Any route of administration may be suitable for the present invention. In one embodiment, the compound of the present invention may be administered to the subject via intravenous injection. In another embodiment, the compounds of the present invention may be administered to the subject via any other suitable systemic deliveries, such as oral, parenteral, intranasal, sublingual, rectal, or transdermal administrations.

In another embodiment, the compounds of the present invention may be administered to the subject via nasal systems or mouth through, e.g., inhalation.

In another embodiment, the compounds of the present invention may be administered to the subject via intraperitoneal injection or IP injection.

As used herein, the term “intraperitoneal injection” or “IP injection” refers to the injection of a substance into the peritoneum (body cavity). IP injection is more often applied to animals than to humans. In general, IP injection may be preferred when large amounts of blood replacement fluids are needed, or when low blood pressure or other problems prevent the use of a suitable blood vessel for intravenous injection.

In animals, IP injection is used predominantly in veterinary medicine and animal testing for the administration of systemic drugs and fluids due to the ease of administration compared with other parenteral methods.

In humans, the method of IP injection is widely used to administer chemotherapy drugs to treat some cancers, in particular ovarian cancer. Although controversial, this specific use has been recommended as a standard of care.

Certain compounds contained in compositions of the present invention may exist in particular geometric or stereoisomeric forms. In addition, polymers of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

For purposes of this invention, the chemical elements are identified in accordance with the Periodic Table of the Elements, CAS version, “Handbook of Chemistry and Physics”, 67th Ed., 1986-87, inside cover.

As used herein, the term “subject” or “individual” refers to a human or other vertebrate animal. It is intended that the term encompass “patients.”

The term “synergistic” refers to a combination which is more effective than the additive effects of any two or more single agents. A synergistic effect permits the effective treatment of a disease using lower amounts (doses) of individual therapy. The lower doses result in lower toxicity without reduced efficacy. In addition, a synergistic effect can result in improved efficacy. Finally, synergy may result in an improved avoidance or reduction of disease as compared to any single therapy.

Combination therapy can allow for the product of lower doses of the first therapeutic or the second therapeutic agent (referred to as “apparent one-way synergy” herein), or lower doses of both therapeutic agents (referred to as “two-way synergy” herein) than would normally be required when either drug is used alone.

As used herein, “pharmaceutically-acceptable carrier” includes any and all solvents, dispersion media, coatings, antibacterial and antifungal agents, isotonic agents, absorption delaying agents, and the like. Pharmaceutically-acceptable carriers are materials, useful for the purpose of administering the compounds in the method of the present invention, which are preferably non-toxic, and may be solid, liquid, or gaseous materials, which are otherwise inert and pharmaceutically acceptable, and are compatible with the compounds of the present invention. Examples of such carriers include oils such as com oil, buffers such as PBS, saline, polyethylene glycol, glycerin, polypropylene glycol, dimethylsulfoxide, an amide such as dimethylacetamide, a protein such as albumin, and a detergent such as Tween 80, mono- and oligopolysaccharides such as glucose, lactose, cyclodextrins and starch.

The formulation used in the present invention may also contain stabilizers, preservatives, buffers, antioxidants, or other additives known to those of skill in the art. The use of such media and agents for pharmaceutically-active substances is well known in the art. Supplementary active compounds can also be incorporated into the imaging agent of the invention. The imaging agent of the invention may further be administered to an individual in an appropriate diluent or adjuvant, co-administered with enzyme inhibitors or in an appropriate carrier such as human serum albumin or liposomes. Pharmaceutically-acceptable diluents include sterile saline and other aqueous buffer solutions. Adjuvants contemplated herein include resorcinols, non-ionic surfactants such as polyoxyethylene oleyl ether and n-hexadecyl polyethylene ether. Enzyme inhibitors include pancreatic trypsin inhibitor, diethylpyrocarbonate, and trasylol. Liposomes inhibitors include water-in-oil-in-water CGF emulsions, as well as conventional liposomes (see J. Neuroimmunol. 1984, 7, 27).

As described herein, certain embodiments of the present compounds may contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts and the like. See J. Pharm. Sci. 1977, 66, 1-19.

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, e.g., J. Pham. Sci. 1977., supra)

More specifically, the compounds that can be formulated into a pharmaceutical composition include a therapeutically-effective amount of the first compound, a therapeutically effective amount of the second compound, and a pharmaceutically-acceptable carrier. The therapeutically-effective amount of the compounds and the specific pharmaceutically-acceptable carrier will vary depending upon, e.g., the age, weight, sex of the subject, the mode of administration, and the type of viral condition being treated.

In a particular aspect, the pharmaceutical composition which can be used includes the compounds of the present invention in effective unit dosage form. As used herein, the term “effective unit dosage” or “effective unit dose” is used herein to mean a predetermined amount sufficient to be effective against AD or the like. Examples include amounts that enable treatment of amyloid deposit(s) in vivo or in vitro that yield acceptable toxicity and bioavailability levels for pharmaceutical use, and/or prevent cell degeneration and toxicity associated with fibril formation.

The pharmaceutical compositions may contain the first compound or the second compound used in the method of this invention in an amount of from 0.01 to 99% by weight of the total composition, preferably 0.1 to 80% by weight of the total composition. For oral administration, the first compound or the second compound is generally administered in an amount of 0.1 g/body to 15 g/body, preferably 0.5 g/body to 5 g/body. For intravenous injection, the dose may be about 0.1 to about 30 mg/kg/day, preferably about 0.5 to about 10 mg/kg/day. If applied topically as a liquid, ointment, or cream, the first compound or the second compound may be present in an amount of about 0.1 to about 50 mg/mL, preferably about 0.5 to 30 mg/mL of the composition.

For systemic administration, the daily dosage as employed for adult human treatment will range from about 0.1 mg/kg to about 150 mg/kg, preferably about 0.2 mg/kg to about 80 mg/kg.

All of the U.S. patents and U.S. patent application publications cited herein are hereby incorporated by reference.

Those skilled in the art will recognize, or be able to ascertain using no more than routine experimentation, many equivalents to the specific embodiments of the invention described herein. Such equivalents are intended to be encompassed by the following claims.

### Method of Treatment.

Combinations of the compounds described above may be administered to a subject in a single dosage form or by separate administration of each active agent. The agents may be formulated into a single tablet, pill, capsule, or solution for parenteral administration and the like. Individual therapeutic agents may be isolated from other therapeutic agent(s) in a single dosage form. Formulating the dosage forms in such a way may assist in maintaining the structural integrity of potentially reactive therapeutic agents until they are administered. Therapeutic agents may be contained in segregated regions or distinct caplets or the like housed within a capsule. Therapeutic agents may also be provided in isolated layers in a tablet.

Alternatively, the therapeutic agents may be administered as separate compositions, e.g., as separate tablets or solutions. One or more active agent may be administered at the same time as the other active agent(s) or the active agents may be administered intermittently. The length of time between administrations of the therapeutic agents may be adjusted to achieve the desired therapeutic effect. In certain instances, one or more therapeutic agent(s) may be administered only a few minutes (e.g., about 1, 2, 5, 10, 30, or 60 min) after administration of the other therapeutic agent(s). Alternatively, one or more therapeutic agent(s) may be administered several hours (e.g., about 2, 4, 6, 10, 12, 24, or 36 h) after administration of the other therapeutic agent(s). In certain embodiments, it may be advantageous to administer more than one dosage of one or more therapeutic agent(s) between administrations of the remaining therapeutic agent(s). For example, one therapeutic agent may be administered at 2 hours and then again at 10 hours following administration of the other therapeutic agent(s). The therapeutic effects of each active ingredient should overlap for at least a portion of the duration, so that the overall therapeutic effect of the combination therapy is attributable in part to the combined or synergistic effects of the combination therapy.

The dosage of the active agents will generally be dependent upon a number of factors including pharmacodynamic characteristics of each agent of the combination, mode and route of administration of active agent(s), the health of the patient being treated, the extent of treatment desired, the nature and kind of concurrent therapy, if any, and the frequency of treatment and the nature of the effect desired. In general, dosage ranges of the active agents often range from about 0.001 to about 250 mg/kg body weight per day. For a normal adult having a body weight of about 70 kg, a dosage may range from about 0.1 to about 25 mg/kg body weight. However, some variability in this general dosage range may be required depending upon the age and weight of the subject being treated, the intended route of administration, the particular agent being administered and the like. Since two or more different active agents are being used together in a combination therapy, the potency of each agent and the interactive effects achieved using them together must be considered. Importantly, the determination of dosage ranges and optimal dosages for a particular mammal is also well within the ability of one of ordinary skill in the art having the benefit of the instant disclosure.

Dosage ranges for agents may be as low as 5 ng/d. In certain embodiments, about 10 ng/day, about 15 ng/day, about 20 ng/day, about 25 ng/day, about 30 ng/day, about 35 ng/day, about 40 ng/day, about 45 ng/day, about 50 ng/day, about 60 ng/day, about 70 ng/d, about 80 ng/day, about 90 ng/day, about 100 ng/day, about 200 ng/day, about 300 ng/day, about 400 ng/day, about 500 ng/day, about 600 ng/day, about 700 ng/day, about 800 ng/day, about 900 ng/day, about 1 μg/day, about 2 μg/day, about 3 μg/day, about 4 μg/day, about 5 μg/day, about 10 μg/day, about 15 μg/day, about 20 μg/day, about 30 μg/day, about 40 μg/day, about 50 μg/day, about 60 μg/day, about 70 μg/day, about 80 μg/day, about 90 μg/day, about 100 μg/day, about 200 μg/day, about 300 μg/day, about 400 μg/day, about 500 μg/day, about 600 μg/day, about 700 μg/day, about 800 μg/day, about 900 μg/day, about 1 mg/day, about 2 mg/day, about 3 mg/day, about 4 mg/day, about 5 mg/day, about 10 mg/day, about 15 mg/day, about 20 mg/day, about 30 mg/day, about 40 mg/day, or about 50 mg/day of an agent of the invention is administered.

In certain embodiments, the agents of the invention are administered in pM or nM concentrations. In certain embodiments, the agents are administered in about 1 pM, about 2 pM, about 3 pM, about 4 pM, about 5 pM, about 6 pM, about 7 pM, about 8 pM, about 9 pM, about 10 pM, about 20 pM, about 30 pM, about 40 pM, about 50 pM, about 60 pM, about 70 pM, about 80 pM, about 90 pM, about 100 pM, about 200 pM, about 300 PM, about 400 PM, about 500 pM, about 600 pM, about 700 pM, about 800 pM, about 900 pM, about 1 nM, about 2 nM, about 3 nM, about 4 nM, about 5 nM, about 6 nM, about 7 nM, about 8 nM, about 9 nM, about 10 nM, about 20 nM, about 30 nM, about 40 nM, about 50 nM, about 60 nM, about 70 nM, about 80 nM, about 90 nM, about 100 nM, about 200 nM, about 300 nM, about 400 nM, about 500 nM, about 600 nM, about 700 nM, about 800 nM, or about 900 nM concentrations.

In certain embodiments, the size of the active agent is important. In certain embodiments, the active agent is less than about 3 μm, less than about 2 μm, less than about 1 μm in diameter. In certain embodiments, the active agent is from about 0.1 μm to about 3.0 μm in diameter. In certain embodiments, the active agent is from about 0.5 μm to about 1.5 μm in diameter. In certain embodiments, the active agent is about 0.2 μm, about 0.3 μm, about 0.4 μm, about 0.5 μm, about 0.6 μm, about 0.7 μm, about 0.8 μm, about 0.9 μm, about 1.0 μm, about 1.1 μm, about 1.2 μm, about 1.3 μm, about 1.4 μm, or about 1.5 μm in diameter.

It may be advantageous for the pharmaceutical combination to be comprised of a relatively large amount of the first component compared to the second component. In certain instances, the ratio of the first active agent to second active agent is about 200:1, 190:1, 180:1, 170:1, 160:1, 150:1, 140:1, 130:1, 120:1, 110:1, 100:1, 90:1, 80:1, 70:1, 60:1, 50:1, 40:1, 30:1, 20:1, 15:1, 10:1, 9:1, 8:1, 7:1, 6:1, or 5:1. It further may be preferable to have a more equal distribution of pharmaceutical agents. In certain instances, the ratio of the first active agent to the second active agent is about 4:1, 3:1, 2:1, 1:1, 1:2, 1:3, or 1:4. It also may be advantageous for the pharmaceutical combination to have a relatively large amount of the second component compared to the first component. In certain instances, the ratio of the second active agent to the first active agent is about 30:1, 20:1, 15:1, 10:1, 9:1, 8:1, 7:1, 6:1, or 5:1. In certain instances, the ratio of the second active agent to first active agent is about 100:1, 90:1, 80:1, 70:1, 60:1, 50:1, or 40:1. In certain instances, the ratio of the second active agent to first active agent is about 200:1, 190:1, 180:1, 170:1, 160:1, 150:1, 140:1, 130:1, 120:1, or 110:1. A composition comprising any of the above-identified combinations of first therapeutic agent and second therapeutic agent may be administered in divided doses about 1, 2, 3, 4, 5, 6, or more times per day or in a form that will provide a rate of release effective to attain the desired results. The dosage form may contain both the first and second active agents. The dosage form may be administered one time per day if it contains both the first and second active agents.

For example, a formulation intended for oral administration to humans may contain from about 0.1 mg to about 5 g of the first therapeutic agent and about 0.1 mg to about 5 g of the second therapeutic agent, both of which are compounded with an appropriate and convenient amount of carrier material varying from about 5 to about 95 percent of the total composition. Unit dosages will generally contain between about 0.5 mg to about 1500 mg of the first therapeutic agent and 0.5 mg to about 1500 mg of the second therapeutic agent. The dosage may be about 25 mg, 50 mg, 100 mg, 200 mg, 300 mg, 400 mg, 500 mg, 600 mg, 800 mg, or 1000 mg, etc., up to about 1500 mg of the first therapeutic agent. The dosage may be about 25 mg, 50 mg, 100 mg, 200 mg, 300 mg, 400 mg, 500 mg, 600 mg, 800 mg, or 1 000 mg, etc., up to about 1500 mg of the second therapeutic agent.

In an aspect, the present disclosure relates to embodiments of methods of treatment of neurodegenerative disease in a subject in need thereof, the method comprising administering to the subject an effective dose of a combination of a mast cell stabilizer and a non-steroidal anti-inflammatory drug; wherein the subject is not a carrier for a neurodegenerative disease predisposing genotype.

In any embodiment of the method of treatment, the neurodegenerative disease may be Alzheimer's disease (AD). In any embodiment of the method of treatment, the method may treat ongoing neurodegenerative disease and/or prevent future onset of neurodegenerative disease. In any embodiment, the neurodegenerative disease may, at the time of treatment, be diagnosed or undiagnosed. The neurodegenerative disease may, at the time of treatment, may be early onset. In any embodiment, the mast cell stabilizer may be cromolyn, a cromolyn derivative, a cromolyn homolog, a salt of any of cromolyn, a cromolyn derivative, or a cromolyn homolog, or any combination thereof. In any embodiment, the salt may be a biocompatible salt. In any embodiment, the biocompatible cromolyn salt may be cromolyn sodium.

In any embodiment of the method of treatment, the subject may not be a carrier of any variant of Apolipoprotein E predisposing the subject to onset of Alzheimer's disease. In any embodiment, the subject may not be a carrier of APOE ε4.

In any embodiment of the method of treatment, the cromolyn or biocompatible salt thereof may be formulated as a dry powder for inhalation. In any such embodiment, the cromolyn or biocompatible salt thereof may be formulated for inhalation as a dry powder of less than about 3 microns in particle size.

In any embodiment of the method of treatment, the cromolyn or biocompatible salt thereof may be administered at a dose of about 1 mg/day to about 50 mg/day. In some embodiments, the dose may be about 16 to about 20 mg/day.

1. A method of treatment of neurodegenerative disease in a subject in need thereof, the method comprising:
administering to the subject an effective dose of a combination of a mast cell stabilizer and a non-steroidal anti-inflammatory drug;
wherein the subject is not a carrier for a neurodegenerative disease-predisposing.

2. The method of claim 1, wherein the neurodegenerative disease is Alzheimer's disease.

3. The method of claim 1, wherein the non-steroidal anti-inflammatory drug is ibuprofen.

4. The method of claim 1, wherein the mast cell stabilizer is cromolyn or a biocompatible salt thereof.

5. The method of claim 2, wherein the subject is not a carrier of any variant of Apolipoprotein E predisposing the subject to onset of Alzheimer's disease.

6. The method of claim 5, where in the subject is not a carrier of APOE ε4.

7. The method of claim 4, wherein the cromolyn or a biocompatible salt thereof is cromolyn sodium.

8. The method of claim 4, wherein the cromolyn or biocompatible salt thereof is formulated as a dry powder for inhalation.

9. The method of claim 8, wherein the cromolyn or biocompatible salt thereof is formulated for inhalation as a dry powder of less than about 3 microns in particle size.

10. The method of claim 4, wherein the cromolyn or biocompatible salt thereof is administered at a dose of about 16 mg/day to about 20 mg/day.

11. The method of claim 5, wherein the cromolyn or biocompatible salt thereof is administered at a dose of about 16 mg/day to about 20 mg/day.

12. The method of claim 6, wherein the cromolyn or biocompatible salt thereof is administered at a dose of about 16 mg/day to about 20 mg/day.

13. The method of claim 7, wherein the cromolyn or biocompatible salt thereof is administered at a dose of about 16 mg/day to about 20 mg/day.

14. The method of claim 8, wherein the cromolyn or biocompatible salt thereof is administered at a dose of about 16 mg/day to about 20 mg/day.

15. The method of claim 9, wherein the cromolyn or biocompatible salt thereof is administered at a dose of about 16 mg/day to about 20 mg/day.
